(19) United States
(12) Patent Application Publication
(10) Pub. No.: US 2007/0207490 A1
(43) Pub. Date: Sep. 6, 2007

(54) Method and system for generating sample plate layout for validation

(75) Inventors: Jacquelyn A. Benfield, San Francisco, CA (US); Ravi Gupta, Foster City, CA (US); Ralph Maria Jocham, San Jose, CA (US); Pui-Ling G. Lam, Pacifica, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/714,408

(22) Filed: Mar. 6, 2007

Related U.S. Application Data
(60) Provisional application No. 60/779,537, filed on Mar. 6, 2006.

(51) Int. Cl. C12Q 1/68; G06F 19/00; C12M 3/00

(52) U.S. Cl. 435/6; 435/287.2; 702/20

(57) ABSTRACT

Systems and methods are provided that relate to a platform, techniques, and processes for verifying the precision, sensitivity, accuracy, reproducibility, and other characteristics of biological tests, such as DNA identification or other tests or assays. According to various embodiments, a logic engine can guide a user to arrange, conduct, and record studies designed to ensure that chemistry kits and laboratory operations return dependably valid results. The validation platform can manage the design of the entire validation workflow, from initiation of a verification project for one or more chemistry kits or other assays or equipment, to design of individual studies to test the accuracy, sensitivity, reproducibility, and other parameters of biological testing. The validation engine can automatically generate a sample plate layout to conduct individual assays. The validation engine can likewise automatically generate unified data output recording the studies which were undertaken, the assays used, tests results, and other data.

### RELATED APPLICATION

This application claims priority to U.S. Provisional Application Ser. No. 60/779,537, filed Mar. 6, 2006, entitled “Method and System for Forensic Software Validation”, which provisional application is incorporated by reference in its entirety.

### FIELD

The present teachings relate to the validation of biological tests.

### BACKGROUND

Forensic and other applications can require that the accuracy of biological tests be confirmed, validated, or otherwise verified. The accuracy of forensic identification of DNA samples taken from a crime scene may, for example, need to be confirmed to enter genetic results into a database or into evidence in criminal or other proceedings. When challenged to produce validation of the accuracy or reliability of DNA or other biological testing, state criminal laboratories, commercial laboratories, or other facilities are required to produce records that the chemical tests used to analyze biological material has itself been tested to verify that the assays demonstrate accurate, reproducible results. In the forensics and other communities, establishing that level of validation can require significant resources and a significant amount of time. To plan a validation protocol, generate samples and necessary documentation, perform the necessary tests, analyze those tests, and assemble all data output can require more than a year of time and involve the full or partial attention of multiple laboratory technicians or managers. The design and execution of a full validation protocol on a manual basis, moreover, can lead to errors or incompleteness in testing that leaves results from those assays open to challenge. Other shortcomings in the validation of biological testing exist.

### SUMMARY

The present teachings overcoming these and other problems in the art relate in one regard to systems and methods for validation of biological tests, in which a laboratory technician, manager, or other user can access an integrated validation platform to initiate, research, plan, design, execute, analyze, and record the results of one or more tests. According to various embodiments, the systems and methods can comprise a validation platform or engine which a user can access to initiate, research, plan, design, arrange, perform, analyze, and record the results of tests such as DNA or other assays or tests. According to various embodiments, tests that are required by governing or advisory bodies can be automatically generated, and the user can automatically be presented with a correct series or sequence of test preparations needed to complete a validation or verification study or protocol.

According to various embodiments, the study or protocol can comprise a precision study, a sensitivity study, an accuracy study, a reproducibility study, a mixture study, any combination of those studies, or other studies, protocols, or tests. In some embodiments, the user need not manually or independently consult the standards, chemistries, or criteria for those tests, but instead be presented with that information on an integrated basis. In some embodiments, the validation platform can present the user with the overall testing workflow needed to successfully prepare or complete the validation or verification of a chemistry kit, assay, instrument, or the like. According to various embodiments, the validation platform can present the user with a diagram or other representation of a sample plate layout that can be used to conduct one or more studies or tests. The validation platform can present the user with an output module configured to output or store results of all phases of the validation and/or verification activity, for example recording test data in hard copy or electronic file format. The output and other output or data generated by the validation platform can include statistical information related to identification of DNA fragments or other biological tests or assays.

According to various embodiments, the validation platform can be or include network-enabled resources such as networked computers, databases, or other hardware, software, or resources, or can comprise a stand-alone computer, data store, or other hardware, software, or resources. In some embodiments, a laboratory technician, manager, or other user can access the integrated validation platform to initiate, develop, conduct, complete, and record the history of all phases and aspects of the validation and/or verification of a biological test kit or chemistry.

According to various embodiments, the accuracy, efficiency, and overall turnaround time for producing the verification results for a forensic or other test chemistry can be significantly enhanced.

### DRAWINGS

The present teachings will be described with reference to the accompanying drawings, in which like elements are labeled with like numbers.

FIG. 1 is a flow diagram of showing how an embodiment of the present software extracts validation standards from a governing body.

FIG. 2 illustrates the translation of extracted guidelines to validation tests.

FIG. 3 is a flow diagram of a hierarchical set of validation workflows useful to conduct a validation project, according to various embodiments of the present teachings.

FIGS. 4A-4C are a flow diagram of validation processing, according to various embodiments of the present teachings.

FIG. 5 is a flow diagram of interactions of a validation engine with a series of studies and data storing operations, according to various embodiments of the present teachings.

FIG. 6 is an illustrative arrangement of a set of computing, instrumentation, and other resources for use in validation processing, according to various embodiments of the present teachings.

FIG. 7 illustrates a sample plate loading configurator, according to various embodiments of the present teachings.

FIG. 8 illustrates a validation project output module, according to various embodiments of the present teachings.

### DETAILED DESCRIPTION

According to various embodiments of the present teachings, in general, systems and methods for verification of biological tests can be provided that allow a laboratory technician, manager, or other user or personnel to access a network-enabled validation platform that integrates, manages, and records the activities related to establishing the precision, sensitivity, accuracy, reproducibility, mixture analysis, and other characteristics or results of one or more biological tests. According to various embodiments, the biological tests can comprise genetic or other tests used for forensic purposes, tests for missing persons, paternity or maternity testing, general medical testing, or other applications. According to various embodiments, the biological tests can comprise DNA sequencing, polymerase chain reaction, and related tests or assays, such as detecting alleles, SNPs (single nucleotide polymorphisms), STRs (short tandem repeats), RNA tests, mitochondrial DNA sequencing, or other genetic tests, procedures, protocols, or assays. In some embodiments, DNA and/or RNA extraction protocols can be validated.

According to various embodiments, the present teachings can be applied to match criteria, establish mixture performance, establish standard operating procedures and interpretation guidelines, and other characteristics or results of other biological tests, in addition to genetic tests. According to various embodiments, the present teachings can be applied to verify the precision, sensitivity, accuracy, reproducibility, mixture and other performance characteristics derived from specific chemistry kits, tests, analyses, or assays.

According to various embodiments, the present teachings can be applied to verify the precision, sensitivity, accuracy, reproducibility, mixture analysis, and other characteristics of one or more machines employed in the testing protocol. According to various embodiments, the present teachings can be applied to assess or validate, for example, equipment, instrumentation or machines such as, for example, sequence detection systems such as real-time polymerase chain reaction (PCR) or other amplification machines or instruments, automated liquid handlers, capillary electrophoresis (CE) instruments used for genetic analysis or other applications, genetic analyzers, or other hardware. In some embodiments, the present teaching can be used to match criteria, establish standard operating procedures, and establish interpretation guidelines for such instruments.

According to various embodiments, the present teachings can be applied to assess or validate laboratory or other procedures or processes, such as, for example, validation of sample preparation techniques, or proficiency testing to validate the capabilities or competency of laboratory technicians or other personnel in handling and conducting procedures with one or more chemical kits, assays, or equipment. In some embodiments, the validation platform and associated resources of the present teachings can be applied to track and evaluate forensics casework and database samples. In some embodiments, the present teachings can be applied to assess and validate existing or new chemistry kits or assays as they are introduced, such as, for example, newly-developed short tandem repeat (STR) or other genetic or other kits, assays, or tests. In some embodiments, the present teachings can be applied to validate and evaluate quality control checks, measures, or standards used to assess, track, and manage reagents. In some embodiments, the present teachings can be applied to validate and evaluate quality control and performance checks, measures, or standards used to assess, track, and manage instrumentation or machines such as, for example, real-time PCR or other sequence detection systems, automated liquid handlers, and capillary electrophoresis (CE) instruments used for genetic analysis or other applications, or other equipment or hardware.

According to various embodiments, the present teachings can be used to assess and validate other equipment, instrumentation, machines, hardware, software, data stores, or other resources used in conjunction with any of the foregoing or other forensic or other applications.

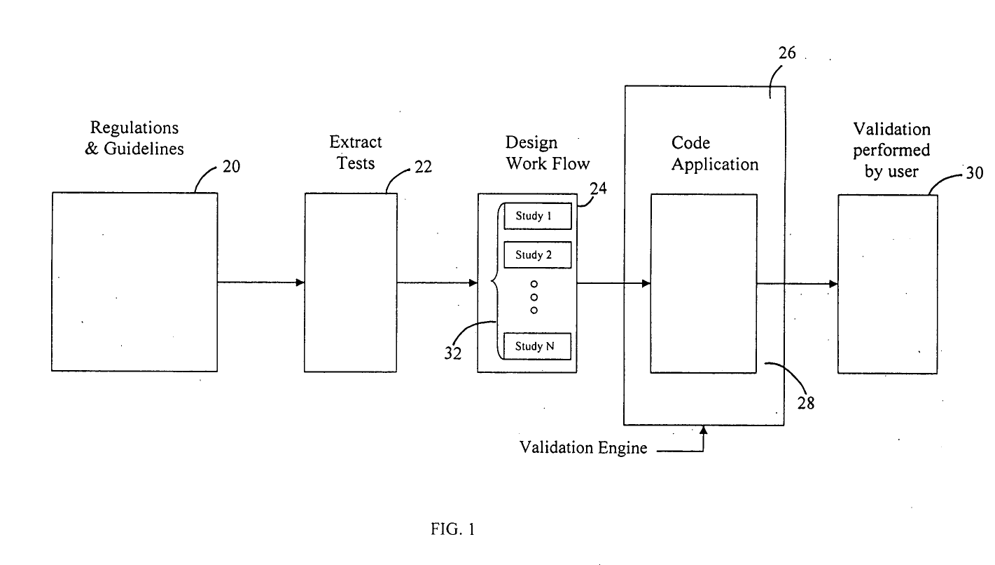

According to various embodiments of the present teachings, a validation engine 26 and associated resources can capture or receive validation standards, guidelines, criteria, and related information to generate a validation project workflow, according for example to the flow diagram of FIG. 1. According to various embodiments as shown in FIG. 1, a set of regulations and guidelines 20, or other validation criteria or information, can be accessed to generate or develop a set of corresponding tests 22. According to various embodiments, the set of regulations and guidelines can be or comprise regulations, guidelines, and other information promulgated, published, transmitted, or otherwise made available by or though governing or advisory bodies, such as those produced by the Scientific Working Group on DNA Analysis Methods (SWGDAM), the National DNA Index System (NDIS), the European Network of Forensic Science Institutes, or other bodies, agencies, organizations, or entities. In some embodiments, the set of regulations and guidelines 20 can be accessed on an automated or other basis, for example by accessing an Internet or other network site for download. In some embodiments, the set of regulations and guidelines can be accessed or received through other channels, connections, or methods, for example, via email transmission, a file transfer protocol (FTP) transmission, delivery of a CD-ROM, or other channels, connections, or other media. In some embodiments, the set of tests 22 can be generated to correspond to various sets of criteria contained in the set of regulations and guidelines 20, for instance, to correspond to requirements related to the precision, sensitivity, accuracy, reproducibility, mixture analysis, or other aspects of a chemical or biological kit, test, assay, or procedure, or hardware, software, or procedures related to the same.

According to various embodiments, the set of tests 22 can be generated automatically, or can be generated manually, or can be generated partly automatically and partly manually, for example with the input of a medical or biological scientist, systems designer, or other personnel. In some embodiments, after a set of tests 22 have been extracted or generated based on the set of regulations and guidelines 20, a validation workflow 24 can be designed. According to various embodiments, the validation workflow 24 can comprise a set of studies 32, for instance, one or more studies related to the precision, sensitivity, accuracy, reproducibility, mixture analysis, or other aspects of a chemical or biological kit, test, assay, or procedure, or associated hardware, software, or procedures. In some embodiments, the set of studies can be generated automatically, manually, or partly automatically and partly manually. In some embodiments, a validation engine 26 can receive, access, or itself generate the set of studies 32 corresponding to the set of regulations and guidelines 20. In some embodiments, validation engine 26 can reside or be hosted in, or interact with, a validation code application 26 and other resources, such as data stores storing the set of regulations and guidelines 20, or other data. In some embodiments, after a suite or set of studies 32 that correspond to the set of regulations and guidelines is generate a user, can perform a validation project 30, for example to validate a chemistry kit used to identify DNA material, or assays used for other purposes. According to various embodiments, the linkage between one or more regulations and guidelines 20 and set of studies 32 accessible via a validation engine 26 or other logic, hardware, or software, can therefore be established for users to conduct a validation study 30, without a need to directly or independently access or consult the set of regulations and guidelines 20, and without a need to attempt to derive the set of tests 22 corresponding thereto. In some embodiments, the set of tests 22 can be updated, automatically or manually, on a regular basis as regulations and guidelines 20 change over time.

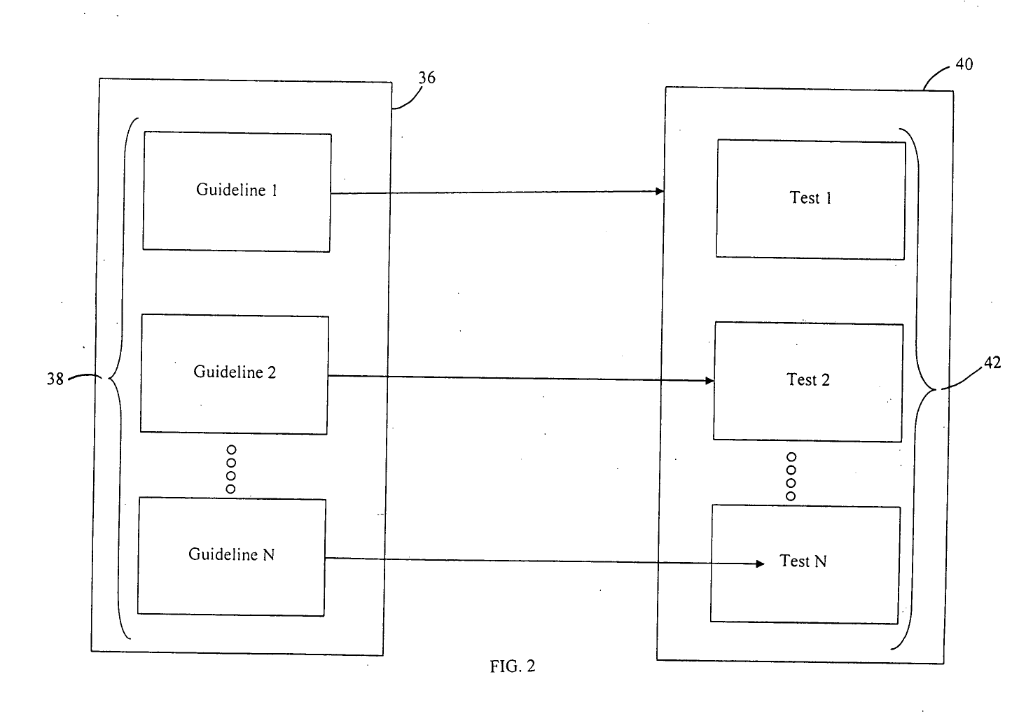

According to various embodiments, and as illustrated in FIG. 2, the regulations and guidelines 20 or other validation information 36 can comprise a set of guidelines 38, such as, for example, threshold or other numerical, statistical, logical, or other criteria regarding the precision, sensitivity, accuracy, reproducibility, mixture analysis, or other aspects of a biological or chemical kit, test, assay, or associated hardware, software, or procedures. According to various embodiments, the set of guidelines 38 can be mapped to or associated with testing information 40 which can comprise a set of tests 42. In some embodiments, each test of the set of tests 42 can correspond to one or more guideline in the set of guidelines, or to other guidelines or criteria. In some embodiments, other couplings or relationships between each test of the set of tests 42 and set of guidelines or other validation information 36 can be established, programmed, or used.

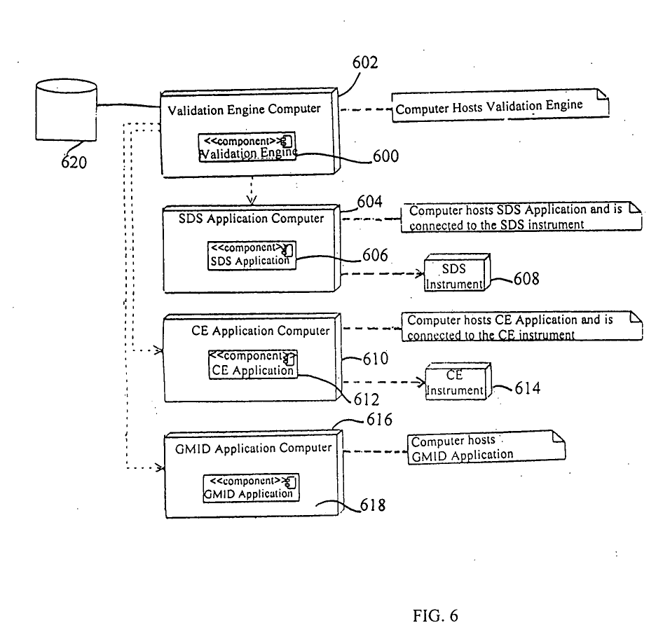

According to various embodiments, and as for instance illustrated in FIG. 6, a laboratory technician, manager, or other user can access a set of computer or other control devices to interact with the validation platform, software, and data stores of the present teachings, and begin a verification project. According to various embodiments as shown, a user can access a validation host computer 602, which can store, run, execute, or otherwise host a validation engine 600. Validation engine 600 can comprise a software application or other programmed logic, storage, or control configured to identify one or more validation workflows necessary to validate and record the proper operation of chemical assays, kits, tests, or analyses, for forensic, medical, or other purposes. According to various embodiments, the validation engine 600 and associate resources can permit a user to identify, plan, prepare, undertake, and record the results of one or more validation tests or studies to satisfy or comply with industry, medical, legal, or other standards or criteria. According to various embodiments, the validation engine 600 can comprise or interface to a validation database 620 or other source of data representing standards, metrics, criteria, or other for establishing the precision, sensitivity, accuracy, reproducibility, and other characteristics of chemistry kits and associated tests performed by a laboratory or other entity. According to various embodiments, the validation engine 600 can communicate or interface with further hardware, software, and instrumentation resources, including, as illustrated, a sequence detection system (SDS) instrument 608 and associated sequence detection system (SDS) host computer 604 and sequence detection system (SDS) application 606, a capillary electrophoresis (CE) instrument 614 and associated capillary electrophoresis (CE) host computer 610 and capillary electrophoresis (CE) application 612, and a genotyping host computer 618 and genotyping application 618. Other instruments, computers, and applications can be accessed and used. According to various embodiments, any one or more of validation host computer 602, sequence detection system (SDS) host computer 604, capillary electrophoresis (CE) host computer 604, genotyping host computer 616, or other machines or hardware can be local or remote, networked by Internet, LAN, or other network, channel or connection, or be configured in stand-alone, distributed, or other arrangements.

According to various embodiments, the validation engine 600 can, for example, store, access, or organize validation and/or verification projects according to standards such as those promulgated by the Scientific Working Group on DNA Analysis Methods (SWGDAM), National DNA Index System (NDIS), European Network of Forensic Science Institutes, the FBI, the ISO, or other organizations or standards. In some embodiments, guidelines and standards developed in the future can be incorporated into the validation engine, workflow planning, and other activities of the validation platform of the present teachings. In some embodiments, later-developed standards can be accessed and incorporated into the validation platform, for example, by automatic download from an Internet or other network site, by manual loading performed by a laboratory technician, manager, or others, or by other connections, channels, methods, or processes. In some embodiments, validation can be performed against more than one standard or set of criteria. In some embodiments, validation can be performed against private or internal standards, rather than, or in addition to, public standards.

According to various embodiments, the validation of a chemistry kit, assay, or other procedure, protocol, equipment, or other aspect of biological testing or analysis according to the present teachings can assist, for example, in ensuring that test results using that chemistry kit or other test or assay can be entered into evidence in legal proceedings, can be recorded in a national database or other database or data store, or otherwise be relied upon as evidence or data. Validation engine 600 and associated resources can conduct a validation project, for example, by automatically identifying, ordering, and organizing a series of test protocols, suites, or studies whose output can confirm that proper and accurate results can be reliably obtained from a chemistry kit, test, assay, hardware, or procedure, as described herein, and can be used to identify and/or establish standard operating procedures and interpretation guidelines.

According to various embodiments, the methods, systems, and software can allow a user to create a validation project. A validation project can comprise the studies and tests needed to validate a specific chemistry kit, assay, instrument, software, or other target. Generally, a new validation project can be created for each target although an existing validation project previously saved can also be opened. In some embodiments, only one validation project can be opened at a time. If the user is working on an existing project, the user can be prompted to save that project before creating a new project. In some embodiments, the user can provide information such as, for example, a project name, the user name, the name or other identification of the reagent or chemistry kit or other entity being validated, and a project description for the validation project report.

According to various embodiments, the methods, systems, and software could provide a validation project comprising at least one or more studies. According to various embodiments, each study could comprise at least one test.

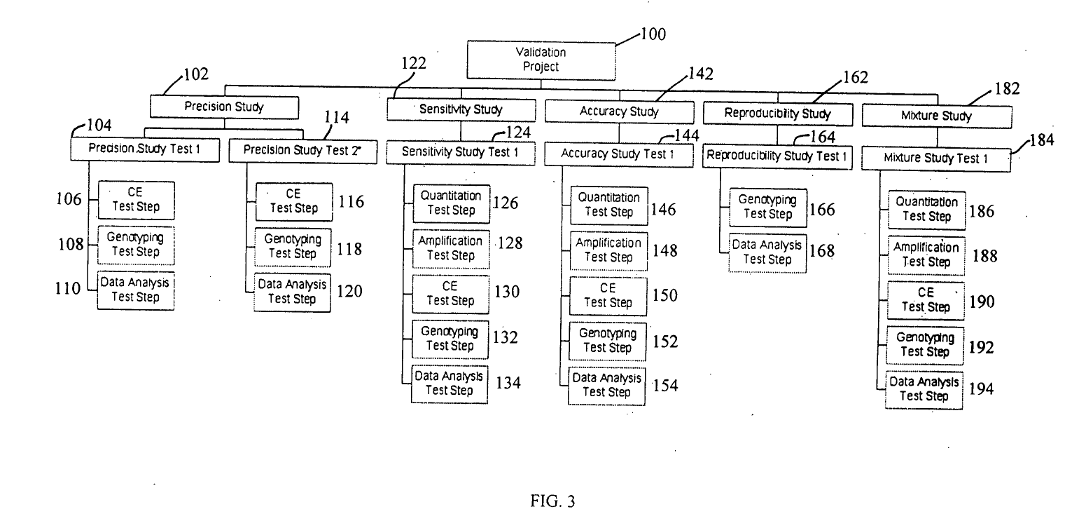

According to various embodiments, and as shown, for example, in FIG. 3, a validation project 100 can comprise five studies: precision study 102, sensitivity study 122, accuracy study 142, reproducibility study 162, and mixture study 182. According to various embodiments, a validation project can comprise a single study, or can comprise two or more studies. According to various embodiments, the number or type of studies to be conducted can be selected or modified by the user. Each study in turn can comprise one or more tests, and each test can comprise one or more test steps needed to meet the study objectives.

According to various embodiments illustrated in FIG. 3, precision study 102 can comprise one or multiple tests, for example two precision study tests 104 and 114 as shown. Precision study test 104 can further comprise capillary electrophoresis test step 106, genotyping test step 108 and/or data analysis test step 110. Similarly, precision study test 114 can further comprise capillary electrophoresis test step 116, genotyping test step 118 and/or data analysis test step 120.

In FIG. 3, sensitivity study 122 can comprise sensitivity study test 124. Sensitivity study test 124 can further comprise quantitation test step 126, amplification test step 128, capillary electrophoresis test step 130, genotyping test step 132, and/or data analysis test step 134.

In FIG. 3, accuracy study 142 can comprise accuracy study test 144. Accuracy study test 144 can further comprise quantitation test step 146, amplification test step 148, capillary electrophoresis test step 150, genotyping test step 152, and/or data analysis step test 154.

In FIG. 3, reproducibility study 162 can comprise reproducibility study test 164. Reproducibility study test 164 can illustratively comprise use of results from accuracy study 142, and can further comprise genotyping test step 166, and/or data analysis test step 168.

In FIG. 3, mixture study 182 can comprise mixture study test 184. Mixture study test 184 can further comprise quantitation test step 186, amplification test step 188, capillary electrophoresis test step 190, genotyping test step 192, and/or data analysis test step 194.

A more detailed description of the various test steps that can used, according to various embodiments, in the various study tests is provided below.

According to various embodiments, the precision study 102 can comprise methods to examine any measurement error that can be inherent or present in a DNA sizing method. According to the embodiment shown in FIG. 3, the precision study can begin with multiple injections into a Genetic Analyzer instrument, a genotyping instrument, or the like, such as, for example, a capillary electrophoresis instrument, of an allelic-ladder from a PCR amplification kit being validated. According to various embodiments, the precision study can examine the degree of precision achieved when sizing an allele multiple times. According to various embodiments, the precision study can characterize the degree of precision, or conversely potential error contributions, from the chemistry kit used for amplification or other reactions, from software used to conduct or analyze the assay, and from the instruments used to conduct the procedure, themselves. After the electrophoresis results are genotyped, the data can be analyzed to verify that the allelic-ladder is genotyped correctly and to calculate the standard deviation of each allele from the allele size. The electrophoresis results can be genotyped using genotyping software such as, for example, GeneMapper® ID, GeneScan®, or Genotyper® software, available from Applied Biosystems, Foster City, Calif. Other genotyping software can be used. The standard deviation and other metrics can be calculated using different calculation methods known in the art.

According to various embodiments, and as shown in FIG. 3, the precision study 102 can be performed first, before the other studies. This can verify the precision of the electrophoresis unit and/or genotyping software being used to validate the PCR amplification kit.

According to various embodiments, and as shown in FIG. 3, the validation project 100 can comprise a sensitivity study 122 that can asses the chemistry, instrument performance, and analysis needed over a range of DNA inputs, the optimal DNA input amount range, and the target DNA input amount, that can be reliably analyzed. The sensitivity study tests 124 can comprise the quantification, preparation, dilution, replication, and/or amplification of quantified DNA samples that are each serially diluted to provide a range of DNA input amounts. After electrophoresis and genotyping of the amplified DNA samples, the data can be analyzed for, for example, genotype concordance, allelic drop-out, peak height, heterozygous peak height ratios, and artifacts. According to various embodiments, the sensitivity study 122 can be performed after the precision study 102. According to various embodiments, the optimal DNA input amounts (the amount that produces the desired peak height) and analysis threshold determined in the sensitivity study 122 can then be used in the accuracy study 142, reproducibility study 162 and mixture study 182.

According to various embodiments, and as shown in FIG. 3, the validation project 100 can further comprise an accuracy study 142 to examine genotyping accuracy and to determine the optimal allele-calling method for the selected PCR amplification kit. Accuracy study test 144 can comprise, for example, quantifying, preparing, diluting, and amplifying a set of quantified DNA samples from known sources in replicate reactions. Then, after electrophoresis and genotyping, the data can be analyzed to calculate genotype concordance and the base pair size of each allele in each unknown sample, and to calculate the deviation from the corresponding allele on the allelic ladder. According to various embodiments, genotype concordance, the base pair size and the deviation can be calculated using one or more different allele-calling methods.

According to various embodiments, the accuracy study 142 can be performed after the precision study 102 and the sensitivity study 122. This can allow the user to utilize the target DNA input amount determined from the sensitivity study 122, and to identify the appropriate allele-calling methods for a particular instrument and laboratory. According to various embodiments, the allele-calling method determined in the accuracy study 142 can then be used in the reproducibility study 162 and mixture study 182.

According to various embodiments, and as shown in FIG. 3, the validation project 100 can further comprise a reproducibility study 162 that can allow the user to evaluate and document the reproducibility of the amplification and genotyping procedures. According to various embodiments, reproducibility study 162 and other studies or tests can share or exchange test steps or results with one or more other studies or tests. According to various embodiments, reproducibility study tests 164 can comprise, for example, the use, extension, or further analysis of results from accuracy study 142, or other studies or tests. According to various embodiments, reproducibility study tests 164 can comprise further additional or independent tests. According to various embodiments, reproducibility study tests 164 can comprise, for example, genotyping the electrophoresis data set generated in the accuracy study 142 using the allele-calling methods determined in the accuracy study 142. After genotyping, the data can be analyzed for genotype concordance, peak height, heterozygous peak height ratios, and artifacts. According to various embodiments, the reproducibility study 162 can be performed after the accuracy study 142 in order to use the data set generated by the accuracy study 142 and to apply the allele calling method determined in the accuracy study 142.

According to various embodiments, and as shown in FIG. 3, the validation project 100 can further comprise a mixture study 182. The mixture study 182 can evaluate mixed DNA samples and can determine the ratios at which a minor contributor profile can reliably be detected. The mixture study tests 184 can involve preparing and analyzing one or more mixtures comprising two quantified DNA samples combined in different ratios. For example, mixture ratios can be created wherein the total amount of genomic input DNA in each mixture is the target DNA input amount determined from the sensitivity study 122 and confirmed in the reproducibility study 162. In another example, mixture ratios can be created wherein each mixture contains a known amount, for example, about 500 ng, of genomic input DNA from a female contributor, plus the amount of male DNA needed to obtain each ratio. According to various embodiments, the mixture study tests 184 can further comprise quantification, mixture preparation, dilution, amplification, electrophoresis, and/or genotyping steps. The data can then be analyzed for, for example, genotype concordance, allele dropout, and individual contributor heterozygous peak height ratios. According to various embodiments, the mixture study 182 can be performed after all other recommended studies, such as for example, precision study 102, sensitivity study 122, accuracy study 142, and reproducibility study 162, in order to use the appropriate target input DNA amounts in the mixed DNA samples, and determine and establish standard operating procedures, analysis thresholds, and interpretation guidelines (identified in the sensitivity study 122).

According to various embodiments, the methods, systems, and software can be used to validate PCR application kits for forensic laboratory applications. Representative amplification kits can be, for example, AmpF1STR® Identifiler®, AmpF1STR® MiniFiler™, and AmpF1STR® Yfiler™ PCR Amplification Kits (Applied Biosystems, Foster City, Calif.).

According to various embodiments, the methods, systems, and software can be used in conjunction with various laboratory instruments and software available from, for example, Applied Biosystems, Foster City, Calif. For quantitation tests the methods, systems, and software can be used in conjunction with, for example, Applied Biosystems quantification kits, ABI PRISM® 7000 Sequence Detection System with SDS software and Applied Biosystems 7500 Real-Time PCR system with SDS software. For amplification tests the method, systems, and software can be used in conjunction with, for example, the GeneAmp® PCR System 9700 thermal cycler and the GeneAmp® PCR System 9600 thermal cycler. For capillary electrophoresis tests, the methods, systems, and software can be used in conjunction with, for example, ABI PRISM® 310 Genetic Analyzer with Data Collection software, ABI PRISM® 3100/3100 Avant™ Genetic Analyzer with Data Collection software, and the Applied Biosystems 3130/3130x1 Genetic Analyzer with Data Collection software. For genotyping tests, various methods, systems, and software according to the present teachings can comprise or can be used in conjunction with, for example, genotyping software such as the aforementioned GeneMapper® ID software, GeneScan® software, and Genotyper® software. Other hardware, software, and assays can be used.

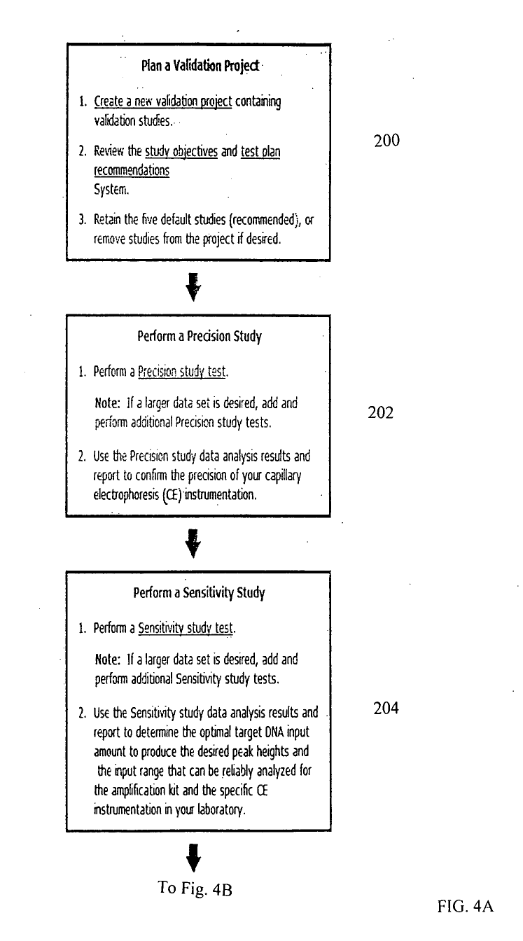

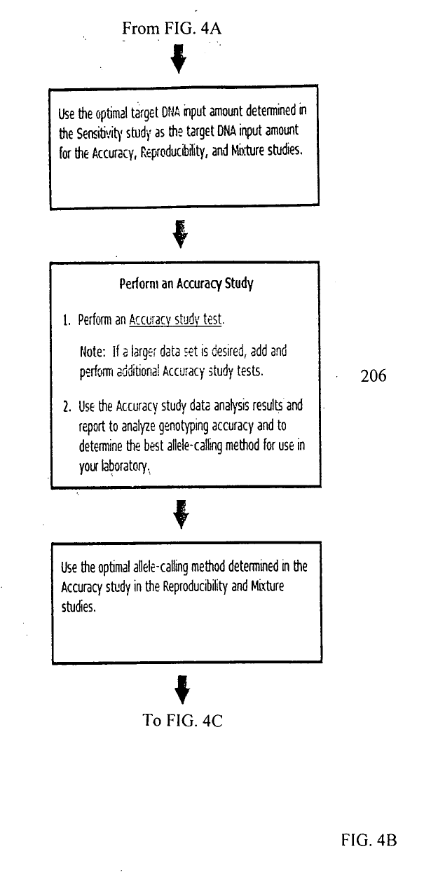

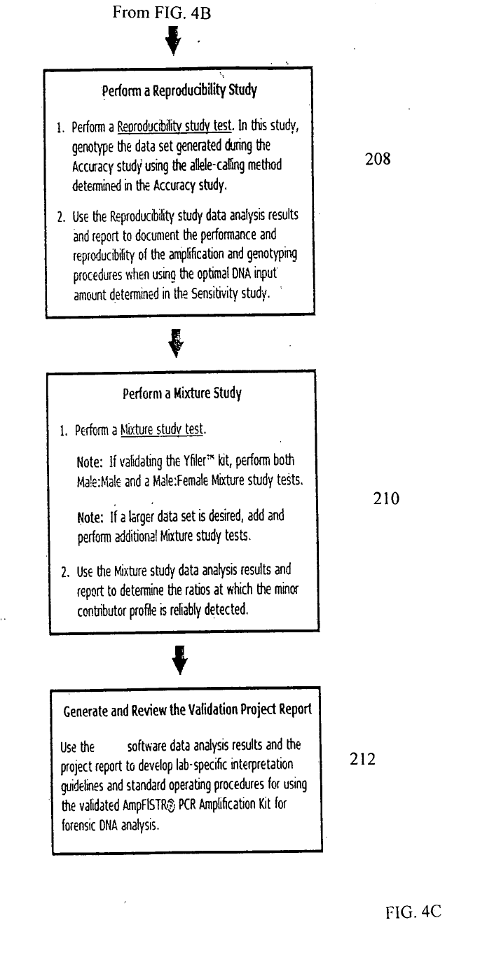

According to various embodiments, the methods, systems, and software can perform the validation studies in a particular sequence. For example, according to various embodiments, and as for example illustrated in FIGS. 4A-4C, the validation project 200 can perform a number, such as five, studies in various orders, including, for example, the following sequence: (a) precision study 202, (b) sensitivity study 204, (c) accuracy study 206, (d) reproducibility study 208, and (e) mixture study 210. After performing the sequence of studies, the validation project can generate a validation project report 212. The validation project 200 shown in FIGS. 4A-4C can generate data analysis results and a project report that can be used to develop lab specific interpretation guidelines and standard operating procedures for PCR amplification kits used in DNA analysis. The guidelines and operating procedures can be used, for example, to validate the AmpF1STR® PCR amplification kit for forensic DNA analysis.

The following examples provide additional guidance and description for illustratively utilizing the methods, systems, and software according to various embodiments of the present teachings. In general, before starting a validation project, a user can prepare the sample reagents, instruments, and software, for instance, according to the manufacture's recommendations. For example, in preparing the reagents the user can order supplies for each test in the project, set these supplies aside, and label them for use in the validation project. The user can also record the lot number of each reagent kit and each individual reagent on a master list that can be referenced for record keeping during each test.

According to various embodiments, reagents with the same lot number can be used for all project tests. In some embodiments, the user can follow the manufacturer's or other suggested storage and shelf life recommendations. In some embodiments, the user can prepare adequate amounts of the quantification standards, such as for example, Quantifiler™ standards (Applied Biosystems, Foster City, Calif.). The user can also prepare adequate buffers, for example T₁₀E_{0.1}, buffer for diluting DNA samples to obtain the target DNA concentration, and capillary electrophoresis running buffer. According to various embodiments, the user can consult or follow the manufacturer's recommendations and directions to become familiar with the chemistry kit, instrument, or software. While certain assays, reagents, and other chemical or biological materials supplied by Applied Biosystems, Foster City, Calif., are illustratively noted herein, it will be appreciated that other assays, reagents, and other chemical or biological materials from that source, or from other manufacturers or sources, can be used and analyzed according to embodiments of the present teachings, singly or in combination.

For preparing the instruments, and before starting each validation project, according to various embodiments, the user can, for example, calibrate one or more instruments. According to various embodiments, the user can record each instrument's serial number, last calibration date, and other information on a master list that can be referenced for record-keeping during each test. According to various embodiments, the methods, systems, and software can include steps to help prepare the real-time PCR instrument, thermal cycler, capillary electrophoresis, or other instrument, instruments, genotyping software, and/or other software, for a validation project. For example, the methods, systems, and software can comprise a checklist that includes steps such as: (a) creating a new matrix or spectral calibration file and recording the file creation date, and (b) beginning a validation project with a new capillary array, new polymer and buffer, clean syringes, and new pump/polymer blocks. The user can also refer to the appropriate instrument user guide for instrument calibration and maintenance procedures. If the user is using a new instrument in the validation project, additional studies may be required to validate the instrument.

According to various embodiments, the user's computer system can be prepared by verifying that certain software or other resources are installed or available, such as, for example, Adobe® Acrobat® Reader® or other software so that the user can view any documents generated in Acrobat (PDF) format. In some embodiments, if needed software is not installed, the user can be directed to locate and install that software, for example by download from an Internet site. In some embodiments, the user can also be directed to import appropriate instrument software, for example to download or import a results group, analysis protocol, protocol for the PCR amplification kit or other chemistry kit, assay, or process being validated. In some embodiments, the user can also import genotyping software table settings and/or table macro files, or other genetic or other information.

### EXAMPLE 1

### Quantitation Plan

According to Example 1, the user can follow the above recommendations for preparing samples, reagents, instruments, and software. Because sample concentration can change over time, the user could quantify samples specifically for each validation project.

Sample and Replicate Counts

The methods, systems, and software can help the user to plan and set up the DNA sample layout. The user can take DNA samples and run them on a Sequence Detection System (SDS) utilizing Applied Biosystems quantification kits. The SDS can quantitate the amount of DNA present in each sample. In some embodiments, analysis tools can be used to provide highlights or flags that indicate the quality and quantity of each sample replicate and standard curve results that enable the user to more quickly evaluate sample quantity and quality. Manual quantitation data entry can also be provided that enables the use of any quantitation method or technology. For example, the data can be used by the system to calculate the minimum volume of sample and diluent needed to run a subsequent test correctly. In some embodiments, dilutions and mixture setup can be included in the calculations.

According to this example, the user could quantify the following number of samples, or a fewer or greater number of samples, including a user-specified number of samples, for each test, although these are exemplary unknowns, the user can quantitate less or more unknowns:

TABLE 1

| | | Number of |
|---|---|---|
| | | Replicates of |
| Sample Type | Number of Samples | Each Sample |
| Unknown | Sensitivity tests: 3 to 5 | 5 |
| | Accuracy/Reproducibility tests: | |
| | 10 to 20 (known samples, non- | |
| | probative case samples, or | |
| | simulated case samples) | |
| | Mixture tests: 3 to 5 | |
| | Note: For Yfiler ™ kit | |
| | validation, quantify both male | |
| | and female DNA samples. | |
| Standards | 8 (set by the software) | 2 |
| Positive Control | no recommendation | no recommendation |
| Negative Control | no recommendation | no recommendation |

### EXAMPLE 2

### Amplification Plan

According to Example 2, the user could follow the above described recommendation for preparing samples, reagents, instruments, and software.

Target DNA Concentrations

According to this example, the user could provide a range of target DNA concentrations that help to identify the optimal target input that produces the desired peak heights.

For example, for sensitivity tests, nine target DNA concentrations could be determined. Exemplary DNA concentrations could be, for example, 4.0, 1.5, 1.25, 1.0, 0.5, 0.25, 0.125, 0.0625, and 0.03125 ng/uL. This set of target DNA concentrations could work with, for example, the Identifiler® PCR amplification kit. A further exemplary set of target DNA concentrations could be: 2.0, 1.5, 1.0, 0.5, 0.25, 0.125, 0.0625, 0.03125, and 0.01560 ng/uL. This set of target DNA concentrations could work with, for example, the Yfiler™, and MiniFiler™ PCR amplification kits. For accuracy/reproducibility and mixture study tests, the user could use the optimal DNA input amount determined with the sensitivity study data set.

Mixture Ratios

For the mixture ratios and the mixture tests, according to this exemplary amplification plan, the user could select three to five mixture sets for each mixture study test. A mixture set could be two DNA samples (or contributors) combined in a number of specific ratios. For example, for Identifiler® and MiniFiler™ PCR amplification kits validation, the contributor volumes could be calculated using the following mixture ratios 1:0, 1:1, 1:3, 1:7, 1:10, 1:15, 1:20,0:1. The user could follow the exemplary mixture set shown below that uses two exemplary DNA samples, 1056D and 1057D.

TABLE 2

| | | DNA Input Amount (ng:ng) | |
|---|---|---|---|
| Mixture Sample | Mixture | Contributor 1 | Contributor 2 |
| Name | Ratio | (Sample 1056) | (Sample 1057) |
| 1056D_1057D_1_0 | 1:0 | 1.0 | 0.0 |
| 1056D_1057D_20_1 | 1:20 | 0.9524 | 0.0476 |
| 1056D_1057D_15_1 | 1:15 | 0.9375 | 0.0625 |
| 1056D_1057D_10_1 | 1:10 | 0.9090 | 0.0910 |
| 1056D_1057D_7_1 | 1:7 | 0.8750 | 0.1250 |
| 1056D_1057D_5_1 | 1:5 | 0.8333 | 0.1667 |
| 1056D_1057D_2_1 | 1:2 | 0.6667 | 0.3333 |
| 1056D_1057D_1_1 | 1:1 | 0.5 | 0.5 |
| 1056D_1057D_0_1 | 0:1 | 0.0 | 1.0 |

For Yfiler kit validation, for example, the user can perform separate mixture study tests to examine male/male and male/female mixtures. For example, a user could use the following mixture sets:
Male:Male test: 1:0, 1:1, 1:3, 1:7, 1:10, 1:15, 1:20,0:1
Male:Female test: 1:0, 1:500, 1:1000, 1:2000, 1:4000, 1:8000, 0:1
   Sample and Replicate Counts

The methods, systems, and software could guide the user to set the number of samples to run in each test step. The user could select samples to fill up several amplification plates per test. For example, the user could amplify the following number of quantified samples, or a fewer or greater number of quantified samples, including a user-specified number of quantified samples, for each test:

TABLE 3

| | | Number of |
|---|---|---|
| | | Replicates of |
| Sample Type | Number of Samples | Each Sample |
| Unknown | Sensitivity tests: 3 to 5 | 3 |
| | Accuracy/Reproducibility tests: | |
| | 10 to 20 (known samples, non- | |
| | probative case samples, or | |
| | simulated case samples) | |
| | Mixture tests: 3 to 5 | |
| Positive Control | 1 per plate | 1 per plate |
| Negative Control* | Identifier ® and MiniFiler ™ kits: | 1 per plate |
| | 1 per plate | |
| | Yfiler ™ kit: 2 per plate | |

*Other numbers or types of controls can be added.

According to various embodiments, the methods, systems, and software could allow the user to review and edit the sample plate layout. For example, according to Example 2, the amplification plate could be created whereby: each column is filled from top to bottom, starting with the left column and moving right, and the unknown samples are placed first, followed by the controls. According to various embodiments, the user can select samples that fill multiple amplification plates per test. According to various embodiments, the user can edit the plate map and other parameters of plate configuration, for example, via a graphical user interface or otherwise. For tests with multiple plates, all replicates of a sample can be placed on the same plate. In sensitivity study tests, all dilutions of a sample can be placed on the same plate. In mixture study tests, samples from the same mixture set could be placed on the same plate. The system can comprise a plate map editing feature to edit any and all plate configurations, for example, quantification, amplification, capillary electrophoresis, and other features can be user editable and configurable.

### EXAMPLE 3

### Capillary Electrophoresis Plan

According to Example 3, the user can follow the above recommendations for preparing samples reagents, instruments, and software. The methods, systems, and software could instruct the user to set up DNA samples for genetic analysis. The DNA samples can be run, for example, on a Genetic Analyzer such as a capillary electrophoresis instrument. The Genetic Analyzer separates and characterizes the DNA in the samples and controls.

Precision Sample and Replicate Counts

A plate could be created for a precision study test, according to Table 4, although less numbers of injections can be used.

TABLE 4

| | | Number of Plate |
|---|---|---|
| Sample Type | Number of Samples | Injections |
| Allelic Ladder | Multiple injections of allelic | Inject the plate |
| | ladder samples are used to evaluate | 6 times |
| | instrument performance. Run the | |
| | allelic ladder provided with the | |
| | target AmpFlSTR ® kit only. | |
| | For 310 (single capillary) | |
| | instruments, 10 replicates of | |
| | allelic ladder | |
| | For 3100/3100-Avant ™ or | |
| | 3130/313Oxl instruments, 16 | |
| | replicates of allelic ladder | |

Sensitivity, Accuracy/Reproducibility, and Mixture Sample and Replicate Counts

According to the embodiment of Example 3, the user can select samples to fill multiple capillary electrophoresis plates per test. The user can run the following number of samples, or a fewer or greater number of samples, including a user-specified number of samples, for each test.

TABLE 5

| | | Number of Plate |
|---|---|---|
| Sample Type | Number of Samples | Injections |
| Unknown | Run amplification replicates of | Inject the |
| | unknown samples from the | plate once |
| | amplification test step. | |
| Allelic Ladder | For 310 (single capillary) | |
| | instruments, every 10th sample | |
| | tube is allelic ladder | |
| | For 3100/3100-Avant ™ or | |
| | 3130/3130xl instruments, every | |
| | 16th well is allelic ladder | |
| Positive | Select 1 positive control from | |
| Control | each amplification plate | |
| | Note: Unknown samples and controls | |
| | from the same amplification plate | |
| | are assigned to the same CE plate. | |
| Negative | Identifier ® and MiniFiler ™ | |
| Control | kits: Select 1 negative control | |
| | from each amplification plate | |
| | Yfiler ™ kit: Select 2 negative | |
| | controls from each amplification | |
| | plate | |
| | Note: Unknown samples and controls | |
| | from the same amplification plate | |
| | are assigned to the same CE plate. | |

### EXAMPLE 4

### Genotyping Plan

According to various embodiments, the data from the Genetic Analyzer could be fed into software that analyzed the data to find the allele location. According to Example 4, the user can follow the system-prepared genotyping worksheet instructions to import sample files from the data collection software, analyze, and genotype the data using, for example, genotyping software.

### EXAMPLE 5

### Data Analysis Plan

According to various embodiments, the output of the Genetic Analysis software could calculate various statistics that help the user to identify samples of interest, identify and establish standard operating procedures, analysis thresholds, and interpretation guidelines, validate the chemistry kit, or point out areas or samples that might need improvement in order for the kit validation to pass. According to various embodiments, a report can be generated and kept on file with the lab to show that they validated the new kit.

According to Example 5, the user can examine the genotyped results with data analysis plots and tables that can variously include, depending on the chemistry kit being validated and other factors, for example:
Precision Study: Concordance and standard deviation
Sensitivity Study: Concordance, allele drop-out, peak height, heterozygote peak height ratios, and artifacts
Accuracy Study: Concordance, sizing deviation
Reproducibility Study: Concordance, peak height, heterozygote peak height ratios, and artifacts
Mixture Study: Concordance, allele drop-out, heterozygote peak height ratios, mixture ratios, and artifacts

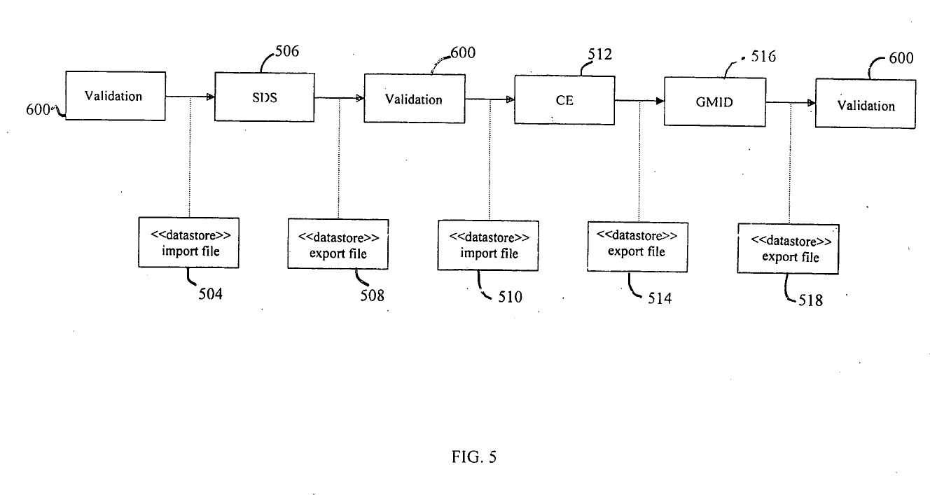

According to various embodiments, generation of a validation plan, sequencing of various studies and tests, and other operations can be automatically executed under control of validation engine 600 and associated logic or control modules and other resources. FIG. 5 illustrates a flow diagram of interactions of validation engine 600 with a series of control modules and data storing operations used to plan, conduct, and record studies and tests for validation purposes. As shown in FIG. 5, according to various embodiments, validation engine 600 can import data from data store 504 in connection with the operation of a sequence detection system (SDS) module 506, to plan and conduct one or more tests or analyses related to genetic analysis. Results of studies and tests identified, organized or conducted by sequence detection system (SDS) module 506 can be exported to data store 508. Control can return to validation engine 600, and validation engine 600 can import data from data store 510 in connection with operation of a capillary electrophoresis (CE) module 512, to plan and conduct one or more tests or analyses related to capillary electrophoresis (CE) separation or other operations. Results of studies and tests identified, organized or conducted by capillary electrophoresis (CE) module 512 can be exported to data store 514. Control can return to validation engine 600, and validation engine 600 can import data from data store 518 in connection with operation of a genotyping software module 516, to plan and conduct one or more tests or analyses related to gene mapping identification or other operations, for instance identification of known or unknown samples using identified alleles. Results of studies and tests identified, organized or conducted by genotyping module 516 can be exported to data store 518. Control can return to validation engine 600, which can initiate or return to further testing, data analysis, or other operations. According to various embodiments, the set of control modules with which validation engine 600 and other resources interact can be extensible. According to various embodiments, the set of control modules can be custom configured by the user, or others.

While description herein of various validation projects, different types of studies within those projects, and tests or assays within individual studies have been illustratively described as occurring in a certain sequence or order, it will be appreciated that according to various embodiments of the present teachings, tests, assays, and studies conducted within individual validation projects, and multiple or related validation projects, can be planned and carried out in different orders or sequences, or omit or add different tests, assays, or studies. For example, according to various embodiments, a mixture study can be conducted before a reproducibility study, or a mixture study can be omitted, or those or any other study or any tests within studies can be omitted or repeated. Other combinations of tests, studies, and validation projects are possible. In some embodiments, for further example, a validation project can comprise an update of or extension to a previous validation project, for example, to incorporate a new study into a completed validation project.

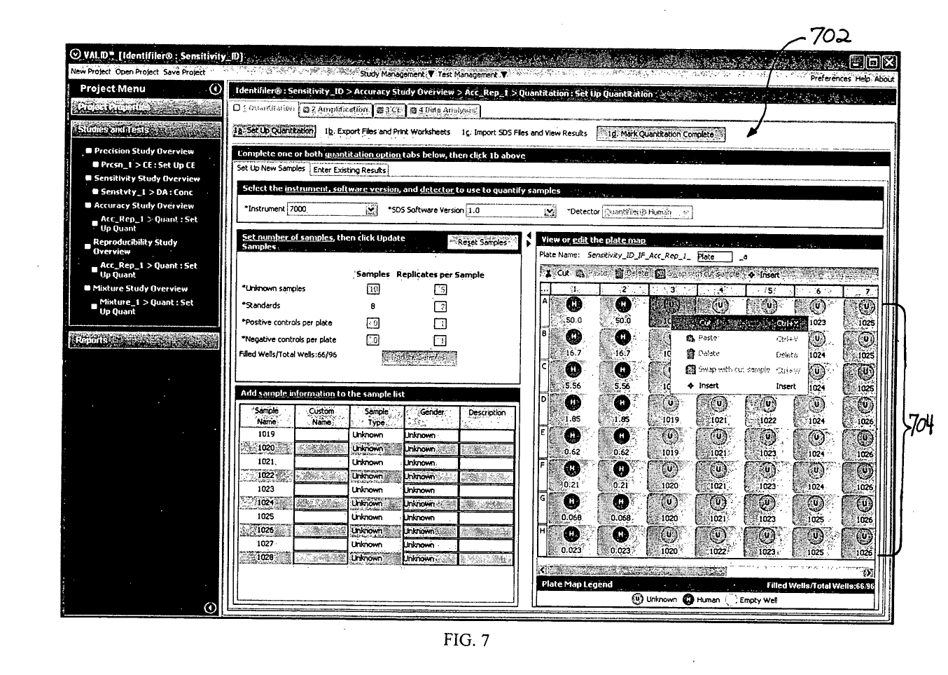

According to various embodiments, for example illustrated in FIG. 7, the validation platform can generate a sample plate configuration 704 necessary to implement the chemical tests, assays, or other procedures that are needed to verify the accuracy and other characteristics of the biological test results. In some embodiments as shown, a user interface 702, such as a graphical user interface, can present the user with a set of sample plate selectors, including a plate view module that displays the sample wells of a standard 96 well, or other plate, holder, or member. In some embodiments, the validation platform can generate a plate configuration 704, such as that shown, to illustrate the physical configuration of a sample plate including sample types, such as DNA samples, standards, and controls, for example, DNA from known human samples (H) and from unknown sources (U) distributed in certain tubes or wells in a 96-well or other sample plate, holder, container, or other member. In some embodiments, the plate configuration 704 can specify or indicate the type of sample that needs to be inserted into individual wells in a plate, the chemistry kit or other assay or test that needs to be applied to a well, group of wells, or entire plate, the concentration of the sample, reagents, or other materials to be loaded into wells of the plate, or other plate configuration and sample parameters. In some embodiments, the plate configuration 704 can lay out the content and sequencing of sample wells in a sample plate for purposes of performing a quantitation test in a sensitivity study, as shown. In some embodiments, the sample plate configuration for other tests and other studies can be generated and represented in plate configuration 704.

According to various embodiments, the plate configuration 704 can be used by a laboratory technician, manager, or other user to manually insert the samples, chemistry kits or other reagents, or other materials to be used in a test or study. According to various embodiments, the plate configuration is also user definable and editable In some embodiments, plate configuration 704 can be used to direct the automatic loading of a sample plate with proper samples and chemistry kits or other reagents, for example using robotic pipettors or other machines operating under program control.

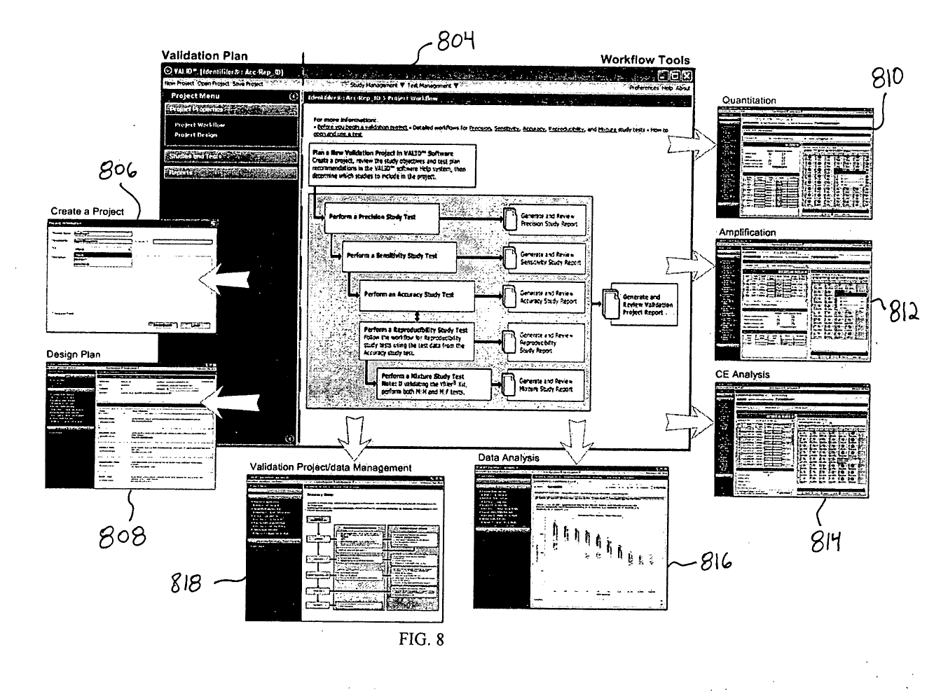

According to various embodiments of the present teachings in a further regard, and as illustrated for example in FIG. 8, the validation platform and associated resources can further generate, produce, store, and output a unified validation project output module 804 that can record or encapsulate various aspects of the validation activity at all stages of the design, testing, and reporting processes. In some embodiments as shown, the validation project output module 804 can comprise data recorded during various studies and other analysis activities. In some embodiments, the validation project output module 804 can generate, capture, or store a separate report, or other section of data, produced by each study. According to various embodiments as shown, the validation project output module 804 can comprise a set of selectable or expandable data reporting modules. In some embodiments as shown, the data reporting modules can comprise, for example, a quantitation data module 810, an amplification data module 812, and a capillary electrophoresis (CE) and data analysis module 814. In embodiments as shown, those data modules can comprise selectable or expandable data sets captured during respective studies of a validation project. According to various embodiments, other types, numbers, or arrangements of data modules extracted or derived from individual test studies can be incorporated in validation project output module 804.

According to various embodiments as likewise illustrated in FIG. 8, the validation project output module 804 can also incorporate other data modules or fields in addition to data modules related to individual studies or tests. For example, validation project output module 804 can comprise a data analysis module 816, such as a set of data that reflects calculated metrics, statistics, or other numerical or logical processing applied to the results from individual test outputs such as quantitation data module 810, amplification data module 812, capillary electrophoresis (CE) and data analysis module 814, or data from other studies or tests. In some embodiments, for example, data analysis module 816 can comprise calculations, metrics, statistics, or other measures such as the standard deviation of study or test results, the least mean squared error around an estimate of study or test results, a linear regression performed on study or test results, a scaling or normalization of study or test results, or other general or specific mathematical or other tests or treatment of any validation data. For example, according to various embodiments, the data analysis module 816 can comprise tests based on or factoring in peak height, peak height ratios, allele stutter percentage estimates, mixture ratios artifact, and other performance parameters. For further example, the data analysis module 816 can contain or comprise comparative data comparing results of a validation project, study, or test against reference profiles or standards, such as Applied Biosystems' standards, private/laboratory references, or standards put forth by governing forensic bodies or other organizations. Other calculations, metrics, data processing, and data output can be performed and stored. In some embodiments, inter study comparisons and collections can be generated to compare data generated across multiple instruments and/or users.

According to various embodiments, the data analysis module 816 can store the results of any such data processing of study or test data, for example in a local or remote database or other storage. According to various embodiments, the data analysis module 816 can execute, for example by user-selectable menus or otherwise, further tests or calculations on any data, to generate new or further data to store, display, or transmit in connection with a validation study or studies. According to various embodiments, the validation project output module 804 can automatically generate the data encapsulated in modules related to individual studies such as quantitation data module 810, amplification data module 812, capillary electrophoresis (CE) data analysis module 814, or others, as well as in data analysis module 816 or other modules, in one or more formats, or combinations of formats. For example, according to various embodiments, data can be stored in a spreadsheet format, in a database format, such as for example in a SQL (standard query language) or other relational or other database host or format, in HTML or XML format, or other formats or representations. In some embodiments, the data formats and the data populated in those formats can be configured to satisfy or conform to standards required or used by validation bodies or organizations. In some embodiments, all data contained in validation project output module 804 can be stored or recorded in a single file or set of related files, in a consistent format. According to various embodiments, difficulties in existing validation practice caused by laboratory personnel having to format, reformat, export and manipulate data from individual studies to execute diverse statistical metrics on the aggregate or comparative data, can be minimized or eliminated.

According to various embodiments, validation project output module 804 can comprise further or additional data derived from other sources, including, as shown, project creation module 806, design plan module 808, and project/data management module 818. In some embodiments, data stored in or processed by those modules can comprise data or metadata related to or associated with initiation, design, implementation, scheduling, and completion of one or more validation projects. That data or metadata can comprise, for example, project timelines, definition of validation goals, progress toward those goals, cost or budget parameters, supply management such as ordering or inventory of chemistry kits, personnel assignments and schedules for persons associated with the one or more validation projects, and other data related to the performance of validation activity.

The foregoing description of systems and methods for verification of biological tests is illustrative, and various alternatives or extensions will occur to persons skilled in the art. For example, according to various embodiments, various computing, instrumentation, software, data storage, and other resources illustrated as singular can be implemented in distributed architectures, and those and other resources illustrated as distributed can be combined.

What is claimed is:

1. A method of generating a configuration for loading a sample plate, comprising:
selecting a chemical assay to be validated;
identifying at least one test to be conducted to validate the chemical assay; and
automatically generating a specification of content to be loaded into at least one sample well of the sample plate to conduct the at least one test.

2. The method of claim 1, wherein the chemical assay comprises a genetic analysis.

3. The method of claim 2, wherein the genetic analysis comprises discriminating at least one allele from at least one biological source.

4. The method of claim 3, wherein the genetic analysis comprises at least two potentially distinct alleles from at least two biological sources.

5. The method of claim 1, wherein the at least one test comprises at least one test associated with at least one of a precision study, a sensitivity study, an accuracy study, a reproducibility study, and a mixture study.

6. The method of claim 1, wherein the content to be loaded into at least one sample well comprises at least one of a predetermined biological sample, at least one primer, at least one probe, and at least one detectable marker.

7. The method of claim 1, wherein the at least one sample well comprises a plurality of sample wells.

8. The method of claim 7, wherein the automatically generating a specification of content comprises automatically generating a respective specification of content for each of the sample wells.

9. The method of claim 8, wherein the specification of content for at least two of the plurality of sample wells is different.

10. The method of claim 1, wherein the validation comprises validation of the chemical assay for use in analyzing forensic evidence.

11. The method of claim 1, further comprising generating an output of the specification of content.

12. The method of claim 11, wherein the output comprises a storable electronic file containing the specification of content.

13. The method of claim 1, further comprising displaying the specification of content to an operator via a graphical user interface.

14. A system for generating a configuration for loading a sample plate, comprising:
an input module for receiving an indication of a chemical assay to be validated and at least one test to be conducted to validate the chemical assay; and
a configuration engine, communicating with the input module, the configuration engine automatically generating a specification of content to be loaded into at least one sample well of the sample plate to conduct the at least one test.

15. The system of claim 14, wherein the chemical assay comprises a genetic analysis.

16. The system of claim 15, wherein the genetic analysis comprises discriminating at least one allele from at least one biological source.

17. The system of claim 16, wherein the genetic analysis comprises at least two potentially distinct alleles from at least two biological sources.

18. The system of claim 14, wherein the at least one test comprises at least one test associated with at least one of a precision study, a sensitivity study, an accuracy study, a reproducibility study, and a mixture study.

19. The system of claim 14, wherein the content to be loaded into at least one sample well comprises at least one of a predetermined biological sample, at least one primer, at least one probe, and at least one detectable marker.

20. The system of claim 14, wherein the at least one sample well comprises a plurality of sample wells.

21. The system of claim 14, wherein the configuration engine is configured to output a storable electronic file containing the specification of content.

22. The system of claim 14, further comprising a graphical user interface configured to display the specification of content to an operator.

23. A sample plate, the sample plate being loaded according to a specification of content generated by a method comprising:
selecting a chemical assay to be validated;
identifying at least one test to be conducted to validate the chemical assay; and
automatically generating a specification of content to be loaded into at least one sample well of the sample plate to conduct the at least one test.

24. The sample plate of claim 23, wherein the chemical assay comprises a genetic analysis.
